(19) United States
(12) Patent Application Publication
(10) Pub. No.: US 2026/0183540 A1
(43) Pub. Date: Jul. 2, 2026

(54) DEFLECTABLE ARTICULATION MEMBER FOR CUTABILITY AN ARTICULATING JOINT DESIGN

(71) Applicant: Cardiac Pacemakers, Inc., St. Paul, MN (US)

(72) Inventors: Andrew L. De Kock, Ham Lake, MN (US); Joseph Geissler, Minneapolis, MN (US); Richhath Ourn, Saint Paul, MN (US); Brent Dolan, Blaine, MN (US)

(21) Appl. No.: 19/432,482

(22) Filed: Dec. 24, 2025

Related U.S. Application Data
(60) Provisional application No. 63/740,236, filed on Dec. 30, 2024.

(51) Int. Cl. A61N 1/05; A61M 25/00

(52) U.S. Cl. CPC A61N 1/056; A61M 25/0015; A61M 2210/125

(57) ABSTRACT

A cuttable articulating catheter assembly comprising a handle member and a catheter shaft extending from the handle member and including an articulation member configured selectively articulate in response to a deflection force, the articulation member including a plurality of tubular sections, and a plurality of joints, wherein each joint of the plurality of joints is positioned between two adjacent tubular sections of the plurality of tubular sections, and wherein a first gap and a second gap are positioned between adjacent sections of the plurality of sections, the first gap positioned on a first side of each joint of the plurality of joints, and the second gap positioned on a second side of each joint of the plurality of joints, and wherein each tubular section includes a cutability feature configured to facilitate cutting of the articulation member along its length.

### CROSS REFERENCE TO PRIOR APPLICATIONS

This application claims priority to U.S. Provisional Patent Application No. 63/740,236, filed December 30, 2024, which is herein incorporated by reference in its entirety.

### TECHNICAL FIELD

The present disclosure relates to catheters that have features therein to help facilitate removal of the catheters. In particular, the present disclosure relates to deflectable catheters with articulating joints that define openings or cutting features.

### BACKGROUND

Medical procedures often require precise placement of leads and other material. For example, conduction system pacing (CSP) leads require placement in the mid septum within the right ventricle. Fixed curved catheters are commonly used, but they are limited in their ability to change in shape to fit in an appropriate location. Deflectable lead delivery catheters allow additional positions such that a physician has more lead placement options. This is particularly valuable in larger distended hearts. Conventional deflectable catheters are not suitable for CSP lead placement.

### SUMMARY

In Example 1, a deflectable lead delivery catheter comprising a handle member and a catheter shaft extending from the handle member. The catheter shaft has a proximal region and a distal region, the distal region including a deflection region, the shaft defining a shaft lumen sized to receive an implantable lead, the deflection region including an articulation member. The articulation member includes a plurality of tubular sections, each section of the plurality of tubular sections defining a cavity extending therethrough, the cavities collectively defining a portion of the shaft lumen, and a plurality of joints, wherein each joint of the plurality of joints is positioned between two adjacent tubular sections of the plurality of tubular sections, and wherein each tubular section of the plurality of tubular sections includes a cutability feature configured to permit selective cutting of the shaft along the articulation member to facilitate removal of the lead delivery catheter following implantation of the implantable lead.

In Example 2, the lead delivery catheter of Example 1, wherein the cutability feature defines a length extending in a first direction that is parallel to a lengthwise direction of the articulation member, and the feature is configured to guide a cutter so that the cutter cuts the articulation member along the lengthwise direction of the articulation member.

In Example 3, the lead delivery catheter of any of Examples 1-2, wherein the cutability feature is an opening in the form of a slot.

In Example 4, the lead delivery catheter of any of Examples 1-2, wherein the cutability feature is a thin wall portion that has a reduced wall thickness relative to other portions of a wall on a respective section.

In Example 5, the lead delivery catheter of any of Examples 1-4, wherein a first auxiliary lumen extends through at least two sections of the plurality of sections.

In Example 6, the lead delivery catheter of Example 5, further comprising a first steering element disposed in the first auxiliary lumen, wherein the first steering element is configured to receive a first tension force to adjust a shape of the articulation member.

In Example 7, the lead delivery catheter of Example 6, wherein the articulation member generally extends along a straight line when no tension is applied to the lead delivery articulation member via the first steering element.

In Example 8, the lead delivery catheter of Example 6, wherein the articulation member has a preformed curvature when no tension is applied to the articulation member via the first steering element.

In Example 9, the lead delivery catheter of any of Examples 5-8, wherein a second auxiliary lumen extends through the at least two sections.

In Example 10, the lead delivery catheter of Example 9, further comprising a second steering element disposed in the second auxiliary lumen, wherein the second steering element is configured to receive a second tension force to adjust the shape of the articulation member.

In Example 11, the lead delivery catheter of any of Examples 9-10, wherein articulation member defines a central axis extending in a lengthwise direction, wherein the lengthwise direction is orthogonal to a first plane, wherein the first auxiliary lumen and the second auxiliary lumen are positioned less than about 180 degrees apart from each other relative to the central axis in the first plane.

In Example 12, the lead delivery catheter of Example 11, wherein the first auxiliary lumen and the second auxiliary lumen are positioned less than about 90 degrees apart from each other relative to the central axis in the first plane.

In Example 13, the lead delivery catheter of any of Examples 1-12, wherein a first gap and a second gap are positioned between adjacent sections of the plurality of sections, the first gap is positioned on a first side of each joint of the plurality of joints, and the second gap is positioned on a second side of each joint of the plurality of joints.

In Example 14, the lead delivery catheter of Example 13, wherein the first gap is smaller than the second gap.

In Example 15, the lead delivery catheter of Example 14, wherein the articulation member is configured to deflect in a first direction in lesser amounts than the articulation member is configured to deflect in a second direction, the first gap decreasing in size when the articulation member deflects in the first direction, and the second gap decreasing in size when the articulation member deflects in the second direction.

In Example 16, a cuttable articulating catheter assembly comprising a handle member, and a catheter shaft extending from the handle member and including an articulation member configured selectively articulate in response to a deflection force. The articulation member includes a plurality of tubular sections, and a plurality of joints. Each joint of the plurality of joints is positioned between two adjacent tubular sections of the plurality of tubular sections, wherein a first gap and a second gap are positioned between adjacent sections of the plurality of sections, the first gap positioned on a first side of each joint of the plurality of joints, and the second gap positioned on a second side of each joint of the plurality of joints, and wherein each tubular section includes a cutability feature configured to facilitate cutting of the shaft along the articulation member.

In Example 17, the cuttable articulating catheter assembly of Example 16, wherein the cutability feature defines a length extending in a first direction that is parallel to a lengthwise direction of the articulation member, and the cutability feature is configured to guide a cutter so that the cutter cuts the articulation member along the lengthwise direction of the articulation member.

In Example 18, the cuttable articulating catheter assembly of Example 17, wherein the cutability feature is an opening in the form of a slot.

In Example 19, the cuttable articulating catheter assembly of Example 17, wherein the cutability feature is a thin wall portion that has a reduced wall thickness relative to other portions of a wall on a respective section.

In Example 20, the cuttable articulating catheter assembly of Example 17, wherein the articulation member includes a first auxiliary lumen extending through at least two tubular sections of the plurality of tubular sections.

In Example 21, the cuttable articulating catheter assembly of Example 20, further comprising a first steering element disposed in the first auxiliary lumen, wherein the first steering element is configured to receive a first tension force to selectively deflect the articulation member.

In Example 22, the cuttable articulating catheter assembly of Example 21, wherein the articulation member generally extends along a straight line when no tension is applied to the lead delivery articulation member via the first steering element.

In Example 23, the lead delivery catheter of Example 21, wherein the articulation member includes a second auxiliary lumen extending through the at least two tubular sections.

In Example 24, the cuttable articulating catheter assembly of Example 23, further comprising a second steering element disposed in the second auxiliary lumen, wherein the second steering element is configured to receive a second tension force to selectively deflect the articulation member.

In Example 25, the cuttable articulating catheter assembly of Example 24, wherein articulation member defines a central axis extending in a lengthwise direction, wherein the lengthwise direction is orthogonal to a first plane, wherein the first auxiliary lumen and the second auxiliary lumen are positioned less than about 180 degrees apart from each other relative to the central axis in the first plane.

In Example 26, thee cuttable articulating catheter assembly of Example 25, wherein the first auxiliary lumen and the second auxiliary lumen are positioned less than about 90 degrees apart from each other relative to the central axis in the first plane.

In Example 27, an articulation member for a deflectable lead delivery catheter, the articulation member comprising a plurality of longitudinally spaced tubular sections, and a plurality of joints, wherein each joint of the plurality of joints is positioned between two adjacent tubular sections of the plurality of tubular sections, and wherein a first gap and a second gap separate adjacent tubular sections on opposite sides of the joints positioned therebetween, and wherein each tubular section includes a cutability feature configured to facilitate cutting of the articulation member along its length.

In Example 28, the articulation member of Example 27, wherein the cutability feature defines a length extending in a first direction that is parallel to a lengthwise direction of the articulation member, and the cutability feature is configured to guide a cutter so that the cutter cuts the articulation member along the lengthwise direction of the articulation member.

In Example 29, the articulation member of Example 28, wherein the cutability feature is an opening in the form of a slot.

In Example 30, the articulation member of Example 28, wherein the cutability feature is a thin wall portion that has a reduced wall thickness relative to other portions of a wall on a respective section.

In Example 31, the articulation member of Example 28, wherein the articulation member includes a first auxiliary lumen extending through at least two tubular sections of the plurality of tubular sections, the first auxiliary lumen being dimensioned to slidably receive a steering element.

In Example 32, a cuttable articulating catheter assembly comprising a handle member, and a catheter shaft extending from the handle member and including an articulation member configured selectively articulate in response to a deflection force. The articulation member includes a plurality of longitudinally spaced tubular sections, and a plurality of joints, wherein each joint of the plurality of joints is positioned between two adjacent tubular sections of the plurality of tubular sections, and wherein a first gap and a second gap separate adjacent tubular sections on opposite sides of the joints positioned therebetween, and wherein each tubular section includes a cutability feature, wherein the cutability feature defines a length extending in a first direction that is parallel to a lengthwise direction of the articulation member, and the cutability feature is configured to guide a cutter so that the cutter can cut the shaft along the articulation member in a lengthwise direction of the articulation member.

In Example 33, the lead delivery catheter of Example 32, wherein the cutability feature is an opening in the form of a slot.

In Example 34, the lead delivery catheter of Example 32, wherein the cutability feature is a thin wall portion that has a reduced wall thickness relative to other portions of a wall on a respective section.

In Example 35, the lead delivery catheter of Example 32, wherein the articulation member includes a first auxiliary lumen extending through at least two tubular sections of the plurality of tubular sections, and wherein the lead delivery catheter further comprises comprising a first steering element disposed in the first auxiliary lumen, wherein the first steering element is configured to receive a first tension force to selectively deflect the articulation member first auxiliary lumen being dimensioned to slidably receive a steering element.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a plan view of a deflectable lead delivery catheter according to embodiments of the present disclosure;

FIG. 1A is a perspective view illustrating an exemplary articulation member incorporated into the deflectable lead delivery catheter of FIG. 1;

FIG. 1B is an enhanced, perspective view illustrating the articulation member of FIG. 1A;

FIG. 1C is a right-side view illustrating the articulation member of FIG. 1A;

FIG. 1D is an enhanced, right-side view illustrating the articulation member of FIG. 1C;

FIG. 1E is a front view illustrating the articulation member of FIG. 1A;

FIG. 1F is a top view illustrating the articulation member of FIG. 1A;

FIG. 2A is a perspective view illustrating another exemplary articulation member for incorporation into the deflectable lead delivery catheter of FIG. 1;

FIG. 2B is an enhanced, perspective view illustrating the articulation member of FIG. 2A;

FIG. 2C is a right-side view illustrating the articulation member of FIG. 2A;

FIG. 2D is an enhanced, right-side view illustrating the articulation member of FIG. 2C;

FIG. 2E is a front view illustrating the articulation member of FIG. 2A;

FIG. 2F is a top view illustrating the articulation member of FIG. 2A;

FIG. 3A is a perspective view illustrating another exemplary articulation member for incorporation into the deflectable lead delivery catheter of FIG. 1;

FIG. 3B is an enhanced, perspective view illustrating the articulation member of FIG. 3A;

FIG. 3C is front view illustrating the articulation member of FIG. 3A;

FIG. 3D is a right-side view illustrating the an alternative articulation member;

FIG. 3E is an enhanced right side view illustrating the articulation member of FIG. 3D;

FIG. 4A is an enhanced, perspective view illustrating another exemplary articulation member for incorporation into the deflectable lead delivery catheter of FIG. 1;

FIG. 4B is an enhanced, right-side view illustrating the articulation member of FIG. 4A;

FIG. 4C is an enhanced, top view illustrating the articulation member of FIG. 4A;

FIG. 4D is a cross-sectional view illustrating a portion of a cross section at a thin wall portion in the articulation member of FIG. 4A;

FIGS. 5A and 5B are enhanced, cross-sectional views of exemplary deflectable lead delivery catheters according to embodiments of the disclosure; and

FIG. 6 is a schematic view illustrating exemplary articulation configurations for various deflectable lead delivery catheters according to embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

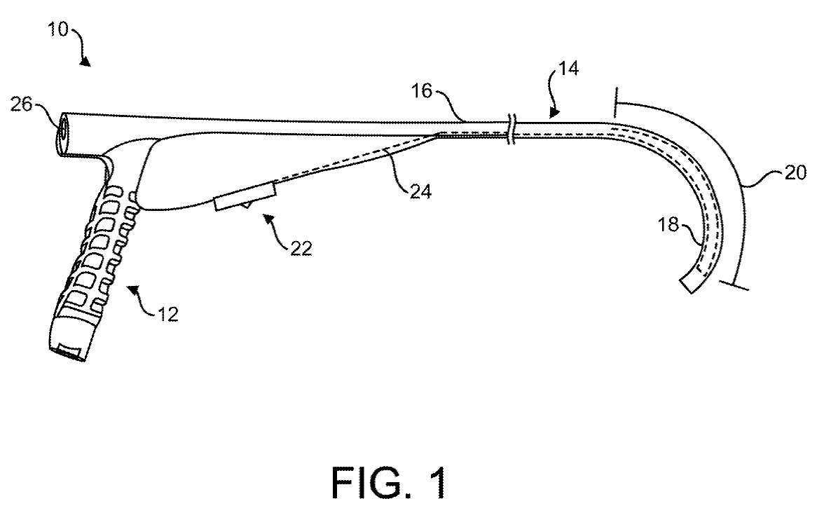

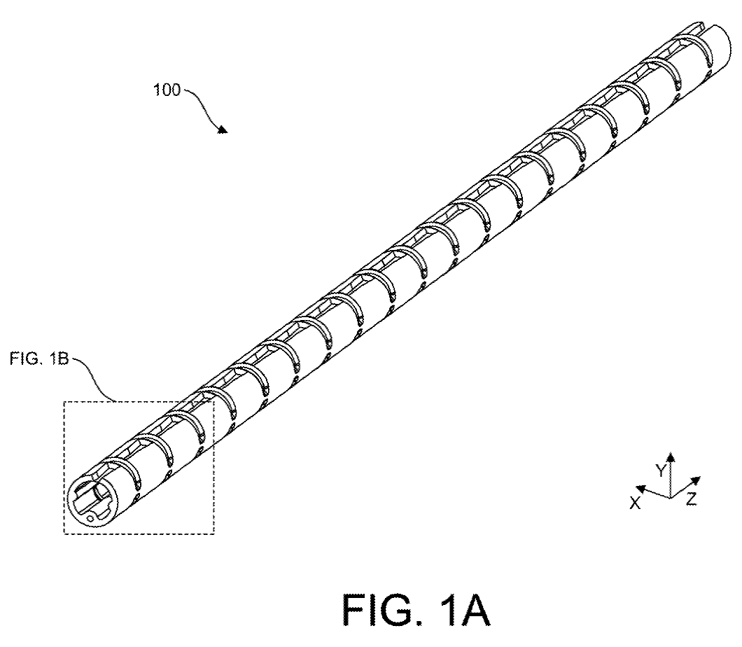

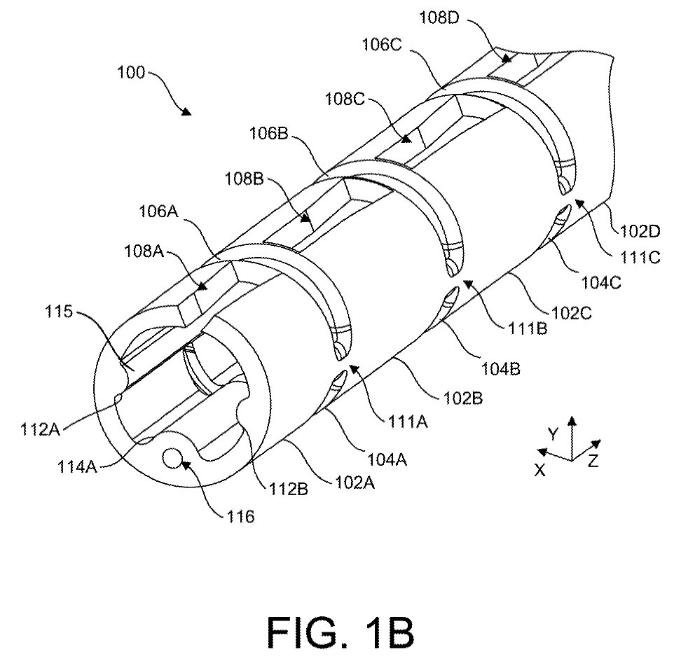

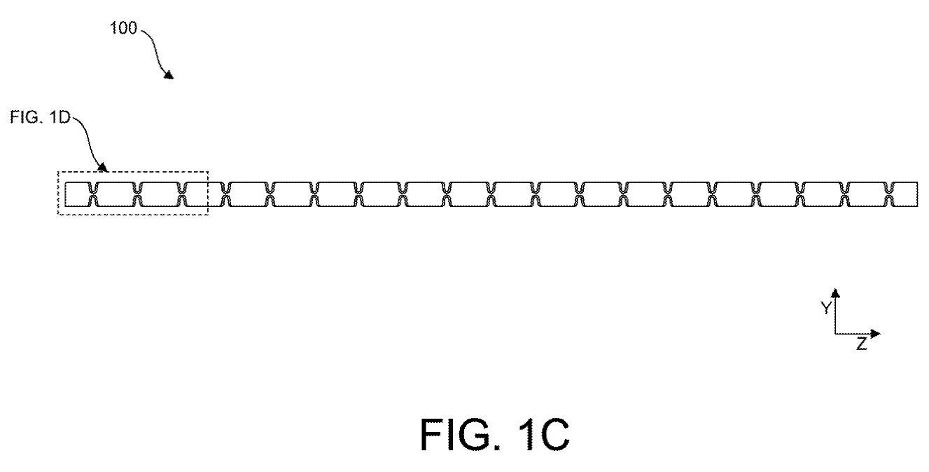

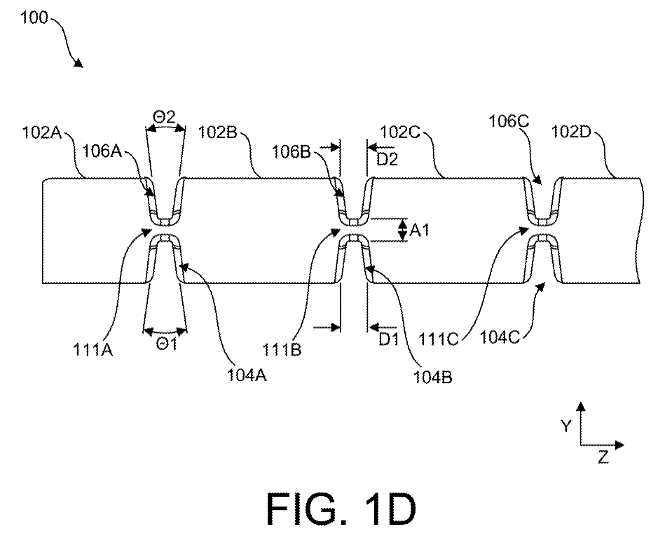

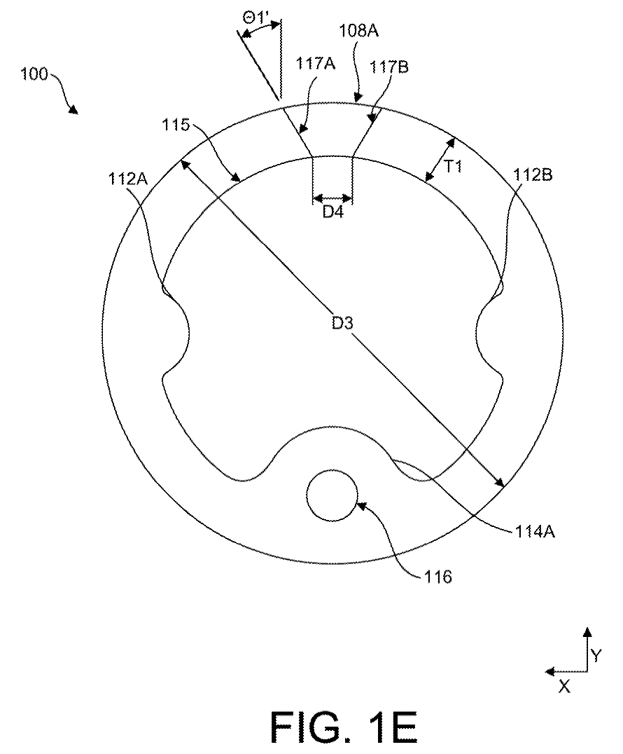

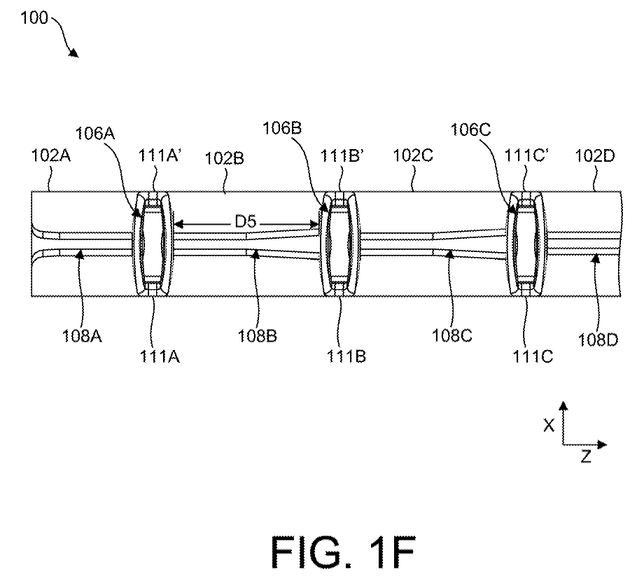

FIG. 1 is a plan view of a deflectable lead delivery catheter 10 that is configured for precise placement of a conduction system pacing (CSP) lead at a target location for selectively pacing one or more of the left bundle branch, the right bundle branch, the His bundle, or other target structures. The particular configuration of the CSP lead is not critical to the present disclosure, and thus the deflectable lead delivery catheter 10 may be used with any number of implantable lead designs.

As shown, the lead delivery catheter 10 includes a handle member 12 and a tubular shaft 14. As further shown, the shaft 14 includes a proximal region 16 and a distal region 18, with the proximal region 16 extending distally from the handle member 12. The distal region 18 further includes a deflection region 20. Additionally, the handle member 12 includes an actuator 22 which is coupled to a steering element 24 that extends through the shaft 14 to a location distal to, or at the distal end of, the deflection region 20. As further shown, a lumen 26 extending from an access opening at the proximal end of the handle 12, and as will be appreciated, extends through the distal end of the shaft 14 such that the shaft 14 has an open distal end to permit deployment of a CSP lead (or other device such as a conventional pacing lead, a guide wire, or another delivery catheter.

The operation of deflectable catheters is well known, and need not be described in great detail herein. In general, the steering element 24 is fixedly attached to the actuator 22, and is further anchored to the shaft 14 at a location distal to, or at the distal end of, the deflection region 20. The actuator 22 can be operated by a user, e.g., by sliding the actuator 22 proximally or distally relative to the handle 12, thereby imparting a deflection force on the shaft 14.

As will be explained in greater detail elsewhere herein, in the various embodiments, the lead delivery catheter 10 includes an articulation member (not shown in FIG. 1) that is specially configured to provide controlled articulation of the catheter 10 when the actuator 22 is manipulated by the user, to enable precise placement of the CSP lead proximate a target location. In particular, the catheter 10 is designed such that the proximal region 16 of the shaft 14 remains relatively undeflected by the operation of the actuator 22, and thus articulation or curvature of the catheter shaft 14 is confined to the deflection region 20. Additionally, as the skilled artisan will recognize, the implantable lead, e.g., CSP lead, deployed through the lumen 26 will include a proximal connector assembly for connection to an implantable pulse generator, wherein the connector assembly has an outer diameter that is greater than the diameter of the lumen 26, which is desirably made as small as possible to inhibit buckling of the lead when it is advanced through the lumen 26. Accordingly, as will be discussed in detail below, the various embodiments of articulation members described herein are particularly configured to permit the catheter shaft 14, including the articulation member, to be slit or cut to facilitate removal of the catheter 10 while leaving the now-deployed lead in place.

FIG. 1A is a perspective view illustrating an exemplary articulation member 100, which may be incorporated in the lead delivery catheter 10 of FIG. 1, according to some embodiments. FIG. 1B is an enhanced perspective view of a portion of the articulation member 100. As shown, the articulation member 100 comprises a plurality of sections. For example, in FIG. 1B, section 102A, section 102B, section 102C, and section 102D are visible. However, additional sections are visible in FIG. 1A. Each of the sections 102A–102D defines a cavity 115 extending through the sections, such that the cavity 115 extends completely through the articulation member 100 along a direction parallel to the Z-axis and corresponds to the catheter lumen 26 shown in FIG. 1. The articulation member 100 is configured to receive a medical device, e.g., a CSP lead body, through the cavity 115.

The sections of the articulation member 100 are connected together via a plurality of joints, and these joints may be articulating joints in some embodiments. For example, section 102A is connected to section 102B via the joint 111A, section 102B is connected to section 102C via the joint 111B, section 102C is connected to section 102D via the joint 111C, and additional sections may similarly be connected via further joints. The articulation member 100 and other articulation members described herein are deflectable articulation members having articulating joints, and these deflectable articulation members may be smoothly used, positioned precisely, and used predictably.

Cutting of a articulation member is often necessary to assist in removal of a articulation member. Each section of the articulation member defines an opening that helps to facilitate cutting of the articulation member. For example, section 102A defines an opening 108A, section 102B defines an opening 108B, section 102C defines an opening 108C, and section 102D defines an opening 108D. While opening 108A extends to the extreme edge of section 102A in the negative Z-direction, sections 102B–102D each include end portions on opposite sides of the openings 108B–108D. The end portions may be beneficial to provide additional strength for the articulation member 100 and to help maintain the shape of the articulation member 100.

Further details regarding the articulation member 100 may be seen in FIGS. 1C to 1D. FIG. 1C is a right-side view illustrating the articulation member of FIG. 1A, and FIG. 1D is an enhanced, right-side view illustrating the articulation member of FIG. 1C, according to some embodiments of this disclosure. As can be seen in FIG. 1D, gaps are positioned on opposing sides of each of the joints. For example, gap 104A is positioned on a first side of the joint 111A, and gap 106A is positioned on a second side of the joint 111A. Gap 104B is positioned on a first side of the joint 111B, and gap 106B is positioned on a second side of the joint 111B. Gap 104C is positioned on a first side of the joint 111C, and gap 106C is positioned on a second side of the joint 111C. Gaps may be positioned similarly relative to other joints. The presence of these gaps allows articulation of one or more sections of the articulation member 100 about the joints. For example, section 102A may be rotated relative to section 102B, with section 102A rotating clockwise or counterclockwise relative to section 102B about a rotational axis that is parallel to the X-axis. Similar movement of different sections may also be accomplished. The permitted amount of rotation may be more limited about rotational axes parallel to the Y-axis as the joints may be more resistant to rotation in this rotational direction. In order to accomplish rotation in a desired direction, the articulation member and/or the shaft within the articulation member may be rotated about the Z-axis relative to the patient, allowing the position of openings and joints to be adjusted. Thus, a wide variety of different shapes may be accomplished for the articulation member 100 and any shaft received therein. The joints and/or the articulation member as a whole may be molded components in some embodiments, but other alternative manufacturing approaches may be used — for example, tubing may be utilized, and slots may be cut in the tubing through laser cutting or mechanical cutting approaches. Other manufacturing techniques may also be used. While gaps are illustrated on both sides, gaps may be formed on only one side in some embodiments. For example, in some alternative embodiments, gaps on one side (e.g., gaps 104A–104C) may be provided and gaps on the opposing side (e.g., gaps 106A–106C) may be omitted.

The articulation member 100 is configured so that the sections may be articulated in equal amounts in two opposing directions. Each of the gaps 104A–104C generally define an angle ϴ1 when the articulation member 100 is in the resting position without any articulation of the articulation member 100 as shown in FIG. 1C and 1D. This angle ϴ1 is measured from the extreme wall of one section to the extreme wall of an opposing section. For example, at gap 104A, the angle ϴ1 is measured from the extreme wall of section 102A next to gap 104A to the extreme wall of section 102B next to gap 104A. Similarly, each of the gaps 106A–106C generally define an angle ϴ2 when the articulation member 100 is in the resting position without any articulation of the articulation member 100 as shown in FIG. 1C and 1D. This angle ϴ2 is measured from the extreme wall of one section to the extreme wall of an opposing section. For example, at gap 106A, the angle ϴ2 is measured from the extreme wall of section 102A next to gap 106A to the extreme wall of section 102B next to gap 106A.

In the articulation member 100, the angle ϴ1 and the angle ϴ2 are approximately the same. However, these angles may differ in other embodiments. Larger values for angles ϴ1, ϴ2 may enable greater articulation as adjacent sections may rotate relative to each other in greater amounts without interfering with each other. Smaller values for angles ϴ1, ϴ2 may allow the amount of articulation in a particular direction to be more limited as adjacent sections may interfere with each other after a lesser amount of articulation, preventing further movement.

Additionally, the gaps 104A–104C each define a distance D1. Distance D1 is measured from points at the extreme edges of adjacent sections, with these points being positioned at the farthest points of the sections along the negative Y-direction. Similarly, gaps 106A–106C each define a distance D2. Distance D2 is measured from points at the extreme edges of adjacent sections, with these points being positioned at the farthest points of the sections along the positive Y-direction. In the articulation member 100, the distances D1 and D2 are equal, but the distances may be different in other embodiments. By changing the distances D1, D2, the amount of possible articulation may be adjusted. For example, lower distances may tend to limit the amount of possible articulation, and larger distances may tend to increase the amount of possible articulation. Each of the joints may also define a thickness in a direction parallel to the Y-axis. For example, the joint 111B defines a thickness A1, and this thickness is about 0.008 inches. Other joints in the articulation member 100 may possess similar thicknesses, and other joints within articulation members of other embodiments may also have a similar thickness. However, the thicknesses of the joints may differ in other embodiments. For example, the thickness A1 could be changed so that it is between about 0.0025 inches and about 0.1000 inches, between about 0.0050 inches and about 0.0090 inches, or between about 0.0070 inches and about 0.090 inches.

Additionally details regarding the articulation member 100 may be seen in the front view of FIG. 1E. The articulation member 100 defines a cavity 115, with this cavity 115 extending all the way through the articulation member 100 along the Z-direction. The articulation member 100 defines a protrusion 112A. This protrusion 112A is positioned at the extreme position of the cavity 115 along the positive X-direction (e.g., the left side of the cavity 115 in FIG. 1E). The articulation member 100 also defines a protrusion 112B positioned at the extreme position of the cavity 115 along the negative X-direction (e.g., the right side of the cavity 115 in FIG. 1E). Protrusions 112A, 112B are positioned approximately 180 degrees apart from each other. However, protrusions may be positioned at other locations within a articulation member in other embodiments, and a greater or lesser number of protrusions may be utilized. Protrusions 112A, 112B may assist with positioning a shaft. While protrusions 112A, 112B do not define openings therein, the protrusions 112A, 112B may define openings therein in other embodiments. In some embodiments, the protrusions 112A, 112B and/or 114A may be omitted.

The surfaces of the sections proximate to openings may also be tapered, and this taper may help ensure that cutting occurs along the opening. For example, in FIG. 1E, a first surface 117A and the second surface 117B are positioned on opposing sides of the opening 108A, with the first surface 117A positioned in the positive X-direction relative to the second surface 117B. Surfaces 117A, 117B each define a taper angle ϴ1’. This taper angle ϴ1’ is about 30 degrees in FIG. 1E, but different taper angles may be used in other embodiments. Alternatively, articulation members may not have tapered surfaces adjacent to openings in other embodiments. The surface 117A and the surface 117B may be separated by a minimum distance D4. The minimum distance D4 determines the thickness of the opening 108A and determines how much the direction of cutting is controlled — the smaller the minimum distance D4, the less freedom of movement that is provided during cutting.

The articulation member 100 also defines a circular shape and a diameter D3. The diameter D3 may vary in different embodiments so that the articulation member 100 may be adapted for different use cases. The diameter D3 may also vary based on the size of the shaft or other objects received within the articulation member 100. Alternatively, the articulation member 200 may define other non-circular shapes in other embodiments.

The articulation member 100 also defines a thickness T1. This thickness T1 is defined at a portion of the articulation member 100 away from the openings, the protrusions 112A, 112B, and the extended portion 114A. In general, the thickness T1 is selected to provide a desired structural characteristics, e.g., to inhibit individual sections from buckling during deflection of the articulation member 100.

The articulation member 100 also defines extended portions at each of the sections. In FIG. 1B, the extended portion 114A is visible at section 102A, but extended portions may be provided in a similar fashion at other sections. The extended portions project into the cavity 115 in a manner similar to protrusions 112A, 112B. In some embodiments, the protrusions 112A, 112B and the extended portion 114A may each contact a shaft extending within the cavity 115 in some embodiments to constrain the motion of the shaft. Additionally, the extended portion 114A may project farther into the cavity 115 than the protrusions 112A, 112B in the articulation member 100, but the relative sizes of the extended portion 114A and the protrusions 112A, 112B may differ in other embodiments.

An auxiliary lumen 116 is also defined within the extended portion of each of the sections of the articulation member 100. The auxiliary lumen 116 may be configured to receive a steering element (see FIG. 1), and tension may be applied to this steering element to cause the shape of the articulation member 100 to be adjusted. In some embodiments, the application of tension on a steering element received at lumen 116 may cause similar amounts of articulation at each of the joints of the articulation member 100, but the amount of articulation in the various joints may differ in other embodiments. In some embodiments, extended portions defining lumens 116 are only positioned at the sections at opposing ends of the articulation member, and a steering element may extend through the lumen at one end section, through the cavity to the opposing end section, and out of the lumen at the opposing end section. However, in other embodiments, extended portions defining lumens 116 may be positioned at one or more sections. In the illustrated embodiment of FIGS. 1A to 1F, the extended portions defining lumens 116 are positioned at each of the sections within the articulation member 100.

FIG. 1F is a top view illustrating the articulation member of FIG. 1A, according to some embodiments of this disclosure. In this top view, the joint 111A’, the joint 111B’, and the joint 111C’ are visible. Each of joints 111A’–111C’ are positioned at the extreme position of the articulation member 100 along the positive X-direction. Joint 111A’ connects section 102A to section 102B, joint 111B’ connects section 102B to section 102C, and joint 111C’ connects section 102C to section 102D. Joints 111A’–111C’ may operate similarly to joints 111A–111C.

Additionally, FIG. 1F allows further details regarding the openings to be seen. Other than openings positioned on sections located at extreme ends of the articulation member 100 (e.g., opening 108A at section 102A), the openings generally define a length D5 along a direction parallel to the Z-axis. The length D5 may vary in different embodiments. Furthermore, the openings may be sized in different proportions relative to the sections in other embodiments. For example, the length of opening 108B may be made smaller to reduce the size of opening 108B relative to the section 102B.

Each of the openings 108A–108D are wider at positions farther in the negative Z-direction, and the openings 108A–108D are narrower at positions farther in the positive Z-direction. This feature may help to guide a cutter as the cutter moves along the positive Z-direction. At the portion of openings 108B–108D that are farther in the negative Z-direction, the openings 108B–108D tend to narrow in width when moving along the positive Z-direction. However, about halfway along the length of the openings 108B–108D, the openings 108B-108D stop narrowing and maintain a constant width. However, this shape for openings 108B–108D is merely exemplary, and other shapes may be used. The articulation member 100 and other articulation members discussed herein may comprise polycarbonate, polypropylene, acetal, nylon, and/or another thermoplastic material, but the articulation members may comprise other materials.

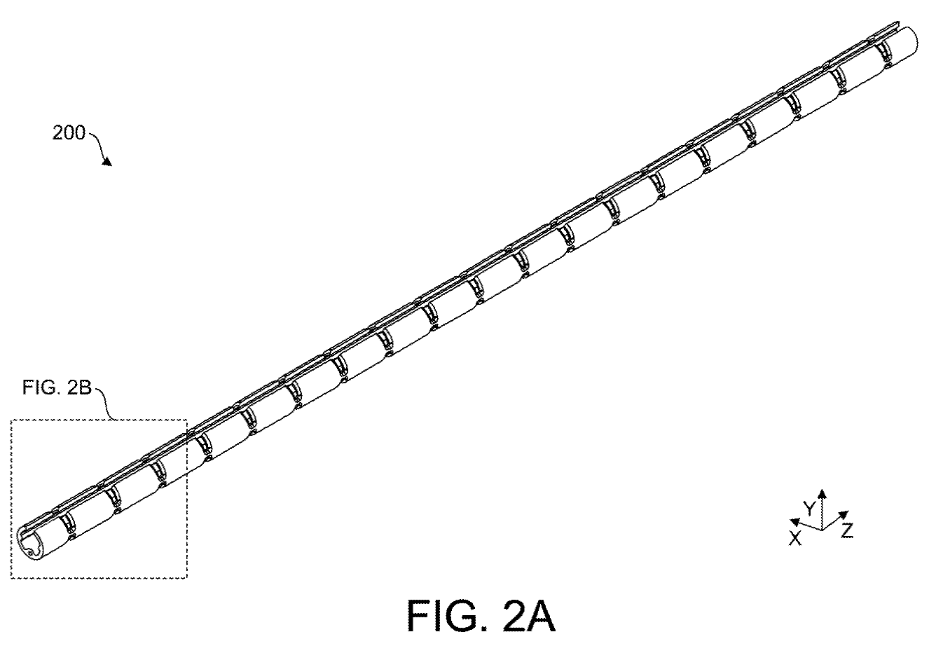

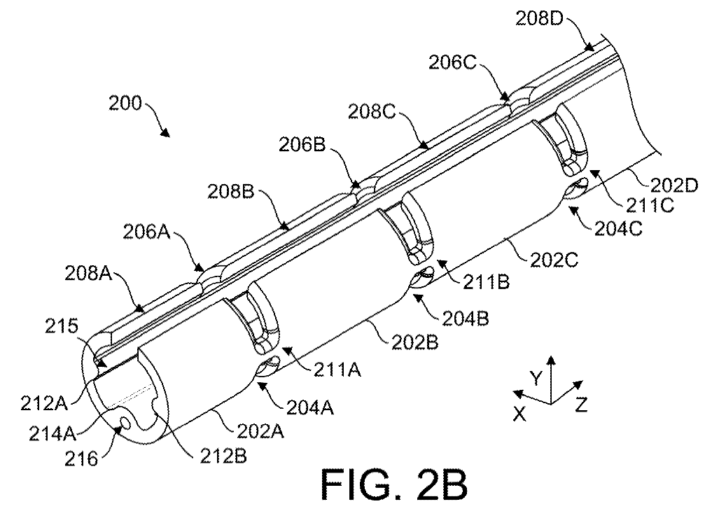

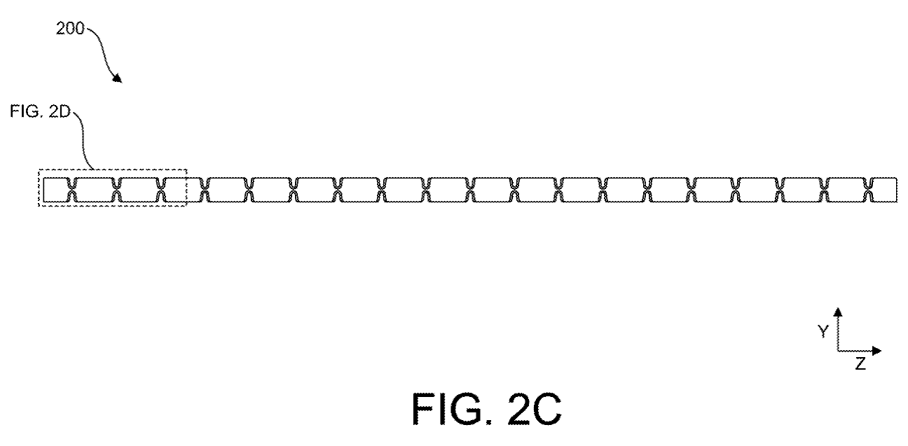

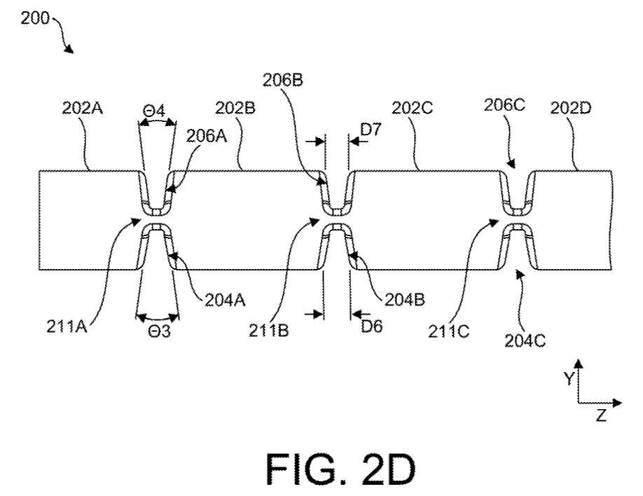

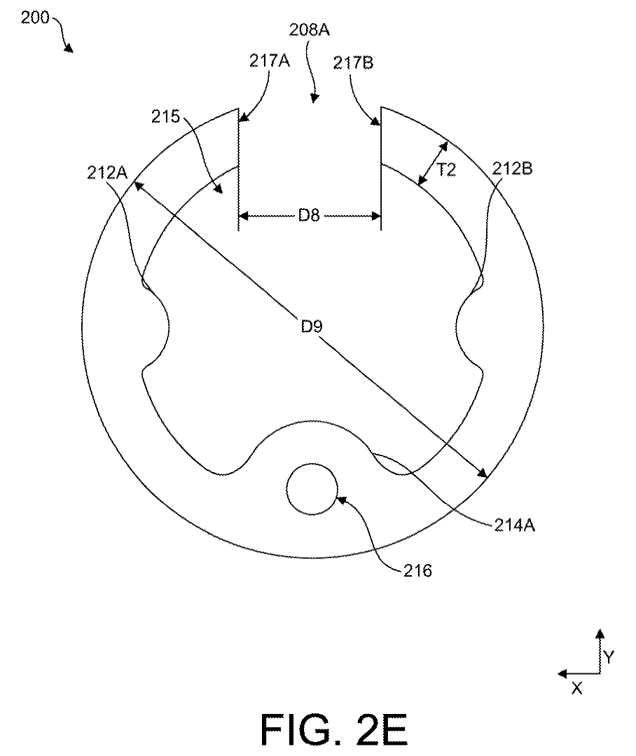

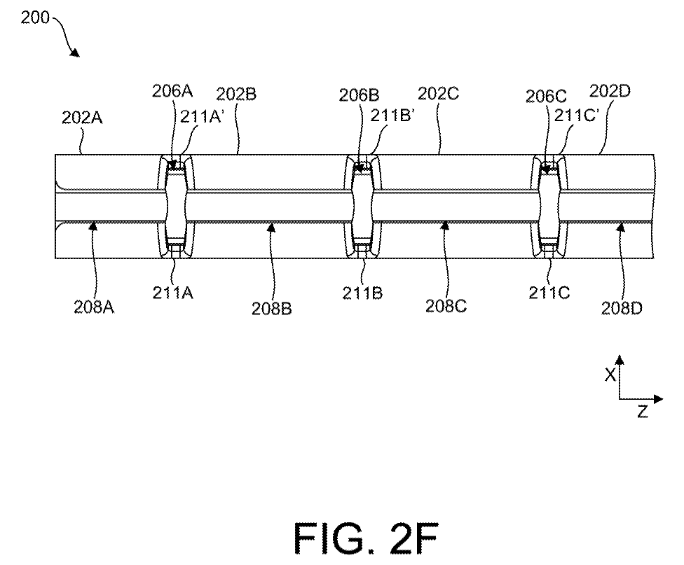

FIG. 2A is a perspective view illustrating an exemplary articulation member 200, which may be incorporated in the lead delivery catheter 10 of FIG. 1, according to some embodiments, and FIG. 2B is an enhanced, perspective view illustrating the articulation member of FIG. 2A. As shown, the articulation member 200 comprises a plurality of sections. For example, in FIG. 2B, section 202A, section 202B, section 202C, and section 202D are visible. However, additional sections are visible in FIG. 2A. Each section of the sections 202A–202D defines a cavity 215 extending through the sections. The cavity 215 extends completely through the articulation member 200 along a direction parallel to the Z-axis, and may correspond to or define the catheter lumen 26 (FIG. 1).

The sections of the articulation member 200 are connected together via a plurality of joints, and these joints may be articulating joints in some embodiments. For example, section 202A is connected to section 202B via the joint 211A, section 202B is connected to section 202C via the joint 211B, section 202C is connected to section 202D via the joint 211C, and additional sections may similarly be connected via further joints. The articulation member 200 and other articulation members described herein are deflectable articulation members having articulating joints, and these deflectable articulation members may be smoothly used, positioned precisely, and used predictably. The joints and/or the articulation member as a whole may be molded components in some embodiments, but other alternative manufacturing approaches may be used — for example, tubing may be utilized, and slots may be cut in the tubing through laser cutting or mechanical cutting approaches. Other manufacturing techniques may also be used. While gaps are illustrated on both sides, gaps may be formed on only one side in some embodiments. For example, in some alternative embodiments, gaps on one side (e.g., gaps 204A–204C) may be provided and gaps on the opposing side (e.g., gaps 206A–206C) may be omitted.

Articulation member 200 has openings in each of the sections, with the openings allowing easy removal of a articulation member without any cutting being required in some embodiments. For example, section 202A defines an opening 208A, section 202B defines an opening 208B, section 202C defines an opening 208C, and section 202D defines an opening 208D. Each of the openings may extend all the way to gaps between the sections, effectively creating a single opening extending in directions parallel to the Z-axis along the articulation member 200.

Further details regarding the articulation member 200 may be seen in FIGS. 2C to 2D. FIG. 2C is a right-side view illustrating the articulation member of FIG. 2A, and FIG. 2D is an enhanced, right-side view illustrating the articulation member of FIG. 2C, according to some embodiments of this disclosure. As can be seen in FIG. 2D, gaps are positioned on opposing sides of each of the joints. For example, gap 204A is positioned on a first side of the joint 211A, and gap 206A is positioned on a second side of the joint 211A. Gap 204B is positioned on a first side of the joint 211B, and gap 206B is positioned on a second side of the joint 211B. Gap 204C is positioned on a first side of the joint 211C, and gap 206C is positioned on a second side of the joint 211C. Gaps may be positioned similarly relative to other joints. The presence of these gaps allows articulation of one or more sections of the articulation member 200 about the joints. For example, section 202A may be rotated relative to section 202B, with section 202A rotating clockwise or counterclockwise relative to section 202B about a rotational axis that is parallel to the X-axis. Similar movement of different sections may also be accomplished.

The permitted amount of rotation may be more limited about rotational axes parallel to the Y-axis as the joints may be more resistant to rotation in this rotational direction. In order to accomplish rotation in a desired direction, the articulation member and/or the shaft within the articulation member may be rotated about the Z-axis relative to the patient, allowing the position of openings and joints to be adjusted. Thus, a wide variety of different shapes may be accomplished for the articulation member 200 and any shaft received therein.

Similar to articulation member 100, the articulation member 200 is configured so that the sections may be articulated in equal amounts in two opposing directions. Each of the gaps 204A–204C generally define an angle ϴ3 when the articulation member 200 is in the resting position without any articulation of the articulation member 200 as shown in FIG. 2C and 2D. This angle ϴ3 is measured from the extreme wall of one section to the extreme wall of an opposing section. For example, at gap 204A, the angle ϴ3 is measured from the extreme wall of section 202A next to gap 204A to the extreme wall of section 202B next to gap 204A. Similarly, each of the gaps 206A–206C generally define an angle ϴ4 when the articulation member 200 is in the resting position without any articulation of the articulation member 200 as shown in FIG. 2C and 2D. This angle ϴ4 is measured from the extreme wall of one section to the extreme wall of an opposing section. For example, at gap 206A, the angle ϴ4 is measured from the extreme wall of section 202A next to gap 206A to the extreme wall of section 202B next to gap 206A.

In the articulation member 200, the angle ϴ3 and the angle ϴ4 are approximately the same. However, these angles may differ in other embodiments. Larger values for angles ϴ3, ϴ4 may enable greater articulation as adjacent sections may rotate relative to each other in greater amounts without interfering with each other. Smaller values for angles ϴ3, ϴ4 may allow the amount of articulation in a particular direction to be more limited as adjacent sections may interfere with each other after a lesser amount of articulation, preventing further movement.

Additionally, the gaps 204A–204C each define a distance D6. Distance D6 is measured from points at the extreme edges of adjacent sections, with these points being positioned at the farthest points of the sections along the negative Y-direction. Similarly, gaps 206A–206C each define a distance D7. Distance D7 is measured from points at the extreme edges of adjacent sections, with these points being positioned at the farthest points of the sections along the positive Y-direction. In the articulation member 200, the distances D6 and D7 are equal, but the distances may be different in other embodiments. By changing the distances D6, D7, the amount of possible articulation may be adjusted. For example, lower distances may tend to limit the amount of possible articulation, and larger distances may tend to increase the amount of possible articulation.

Additional details regarding the articulation member 200 may be seen in the front view of FIG. 2E. The articulation member 200 defines a cavity 215, with this cavity 215 extending all the way through the articulation member 200 along the Z-direction and corresponding to, or defining, the catheter lumen 26 (FIG. 1). The articulation member 200 defines a protrusion 212A. This protrusion 212A is positioned at the extreme position of the cavity 215 along the positive X-direction (e.g., the left side of the cavity 215 in FIG. 2E). The articulation member 200 also defines a protrusion 212B positioned at the extreme position of the cavity 215 along the negative X-direction (e.g., the right side of the cavity 215 in FIG. 2E). Protrusions 212A, 212B are positioned approximately 180 degrees apart from each other, and may function to provide structural strength to the respective joints, e.g., the joint 211A. However, protrusions may be positioned at other locations within a articulation member in other embodiments, and a greater or lesser number of protrusions may be utilized. Alternatively, in embodiments, the protrusions 212A, 212B may be omitted.

The articulation member 200 also defines surfaces at opposing sides of openings. For example, in FIG. 2E, a surface 217A and second surface 217B are positioned on opposing sides of the opening 208A, with the surface 217A positioned in the positive X-direction relative to the surface 217B. Unlike the surfaces 117A, 117B of the articulation member 100, the surfaces 217A, 217B are provided without a taper. However, the articulation member 200 may be modified to included tapered surfaces in place of surfaces 217A, 217B. The surface 217A and the surface 217B may be separated by a minimum distance D8. The minimum distance D8 determines the thickness of the opening 208A and determines how much the direction of cutting is controlled.

The articulation member 200 also defines a circular shape and an outer diameter D9. The diameter D9 may vary in different embodiments so that the articulation member 200 may be adapted for different use cases. The diameter D9 may also vary based on the size of the shaft or other objects received within the articulation member 200. Alternatively, the articulation member 200 may define other non-circular shapes in other embodiments.

The articulation member 200 also defines a thickness T2. This thickness T2 is defined at a portion of the articulation member 200 away from the openings, the protrusions 212A, 212B, and the extended portion 214A. The thickness T2 may be selected to provide a desired structural characteristics.

The articulation member 200 also defines extended portions at each of the sections. An extended portion 214A of section 202A is visible in FIG. 2E, and other extended portions may be positioned similarly on other sections. The extended portion 214A projects radially inward to define the shape of the cavity 215 in a manner similar to protrusions 212A, 212B. Additionally, the extended portion 214A may project farther into the cavity 215 than the protrusions 212A, 212B in the articulation member 200, but the relative sizes of the extended portion 214A and the protrusions 212A, 212B may differ in other embodiments.

An auxiliary lumen 216 is also defined within the extended portion 214A in each of the sections of the articulation member 200. The auxiliary lumen 216 may be configured to receive steering element, e.g., the steering wire 22 of FIG. 1. In some embodiments, extended portions defining lumens 216 are only positioned at the sections at opposing ends of the articulation member, and a wire may extend through the lumen at one end section, through the cavity to the opposing end section, and out of the lumen at the opposing end section. However, in other embodiments, extended portions defining lumens 216 may be positioned at one or more sections. In the illustrated embodiment of FIGS. 2A–2F, the extended portions defining lumens 216 are positioned at each of the sections within the articulation member 200.

FIG. 2F is a top view illustrating the articulation member of FIG. 2A, according to some embodiments of this disclosure. In this top view, the joint 211A’, the joint 211B’, and the joint 211C’ are visible. Each of joints 211A’–211C’ are positioned at the extreme position of the articulation member 200 along the positive X-direction. Joint 211A’ connects section 202A to section 202B, joint 211B’ connects section 202B to section 202C, and joint 211C’ connects section 202C to section 202D. Joints 211A’–211C’ may operate similarly to joints 211A–211C.

Additionally, FIG. 2F allows further details regarding the openings to be seen. For example, opening 208A is wider at positions farther in the negative Z-direction, and opening 208A is narrower at positions farther in the positive Z-direction. This feature may help to guide the cutter as the cutter is first being used at the extreme end of the articulation member 200 along the negative Z-direction. The opening at the opposing end section on extreme end along the positive Z-direction may similarly have a varying width. However, other openings such as openings 208B–208D generally possess a uniform width equal to the minimum distance D8.

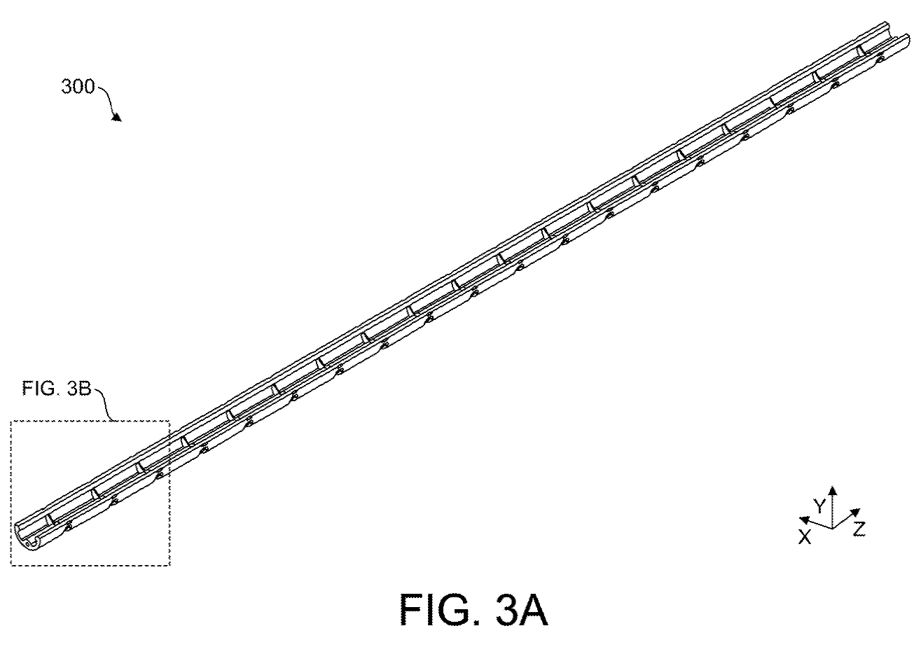

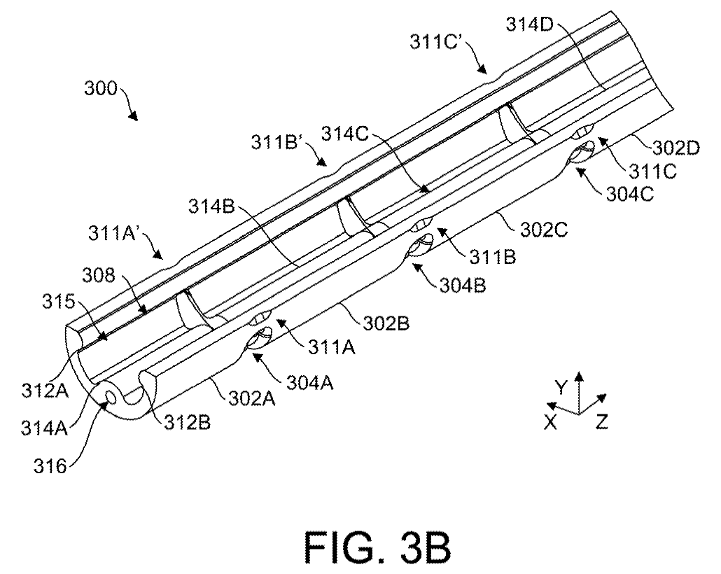

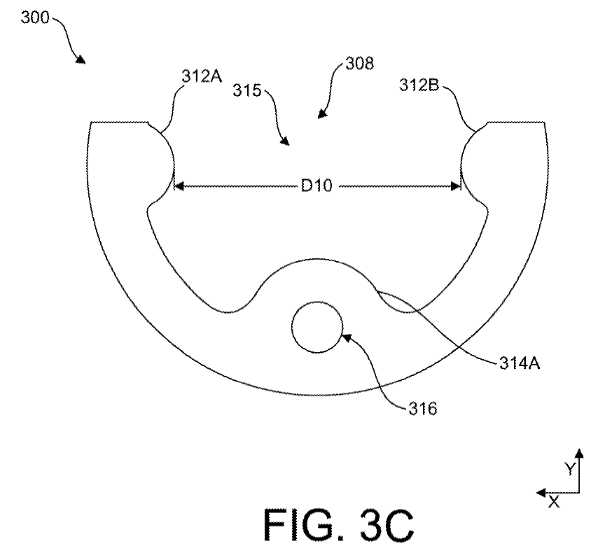

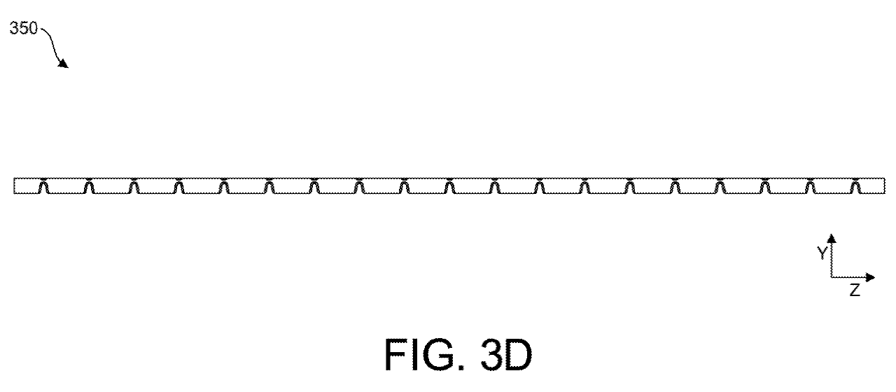

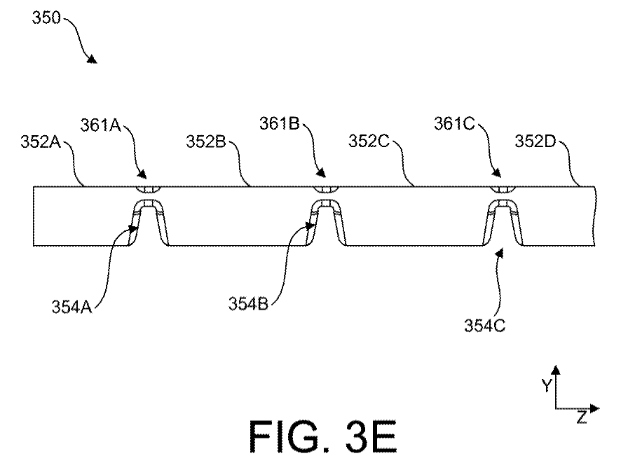

FIG. 3A is a perspective view illustrating another exemplary articulation member 300 , and FIG. 3B is an enhanced, perspective view illustrating the articulation member of FIG. 3A, according to some embodiments of this disclosure. FIG. 3C is an end view of the articulation member 300.

As shown, the articulation member 300 comprises a plurality of sections. For example, in FIG. 3B, section 302A, section 302B, section 302C, and section 302D are visible. However, additional sections are visible in FIG. 3A. Each section of the sections 302A–302D defines a cavity 315 extending through the sections. The cavity 315 extends completely through the articulation member 300 along directions parallel to the Z-axis. The articulation member 300 is configured to at least partially receive a shaft through the cavity 315. The articulation member 300 and other articulation members described herein are deflectable articulation members having articulating joints, and these deflectable articulation members may be smoothly used, positioned precisely, and used predictably.

The sections of the articulation member 300 are connected together via a plurality of joints, and these joints may be articulating joints in some embodiments. For example, section 302A is connected to section 302B via the joint 311A, section 302B is connected to section 302C via the joint 311B, section 302C is connected to section 302D via the joint 311C, and additional sections may similarly be connected via further joints. Similarly, section 302A is connected to section 302B via the joint 311A’, section 302B is connected to section 302C via the joint 311B’, section 302C is connected to section 302D via the joint 311C’. The joints and/or the articulation member as a whole may be molded components in some embodiments, but other alternative manufacturing approaches may be used — for example, tubing may be utilized, and slots may be cut in the tubing through laser cutting or mechanical cutting approaches.

As can be seen in FIG. 3B, a gap is positioned adjacent to each of the joints. For example, gap 304A is positioned adjacent to the joint 311A, gap 304B is positioned adjacent to the joint 311B, and gap 304C is positioned adjacent to the joint 311C. Gaps may be positioned similarly relative to other joints. The presence of these gaps allows articulation of one or more sections of the articulation member 300 about the joints. For example, section 302A may be rotated relative to section 302B, with section 302A rotating clockwise or counterclockwise relative to section 302B about a rotational axis that is parallel to the X-axis. Similar movement of different sections may also be accomplished.

The permitted amount of rotation may be more limited about rotational axes parallel to the Y-axis as the joints may be more resistant to rotation in this rotational direction. In order to accomplish rotation in a desired direction, the articulation member and/or the shaft within the articulation member may be rotated about the Z-axis relative to the patient, allowing the position of openings and joints to be adjusted. Thus, a wide variety of different shapes may be accomplished for the articulation member 300 and any shaft received therein.

The articulation member 300 differs from the aforementioned embodiments in that, as can be seen in the end view of FIG. 3C, it has a generally semi-circular profile when viewed along its longitudinal axis. As shown, the articulation member 300 defines a protrusion 312A. This protrusion 312A is positioned at the extreme position of the cavity 315 along the positive X-direction (e.g., the left side of the cavity 315 in FIG. 3E). The articulation member 300 also defines a protrusion 312B positioned at the extreme position of the cavity 315 along the negative X-direction (e.g., the right side of the cavity 315 in FIG. 3E). Protrusions 312A, 312B are positioned approximately 180 degrees apart from each other, and when present can function to reinforce the respective joints 311A/B/C. As can be seen, the distance D10 between the protrusions 312A, 312B generally defines a relatively large lateral space to facilitate removal of the corresponding delivery catheter from the implanted CSP lead.

FIGS. 3D and 3E are side illustrations of an alternative articulation member 350 according to embodiments of the disclosure. Similar to other embodiments, the articulation member 350 includes a series of sections 352A, 352B, 302C, and 352D, with adjacent sections 352A and 352B connected by joints 361A and 361A’, adjacent sections 352B and 352C connected by joints 361B and 361B’, and adjacent sections 352C and 352D connected by joints 361C and 311C’ so as to define gaps 354A, 354B and 354C, respectively. The articulation member 350 differs from the previously-described embodiments in that the joints connecting adjacent sections, e.g., joints 311A and 311A’, 311B and 311B’, and 311C and 311C’ are not positioned diametrically across from each other.

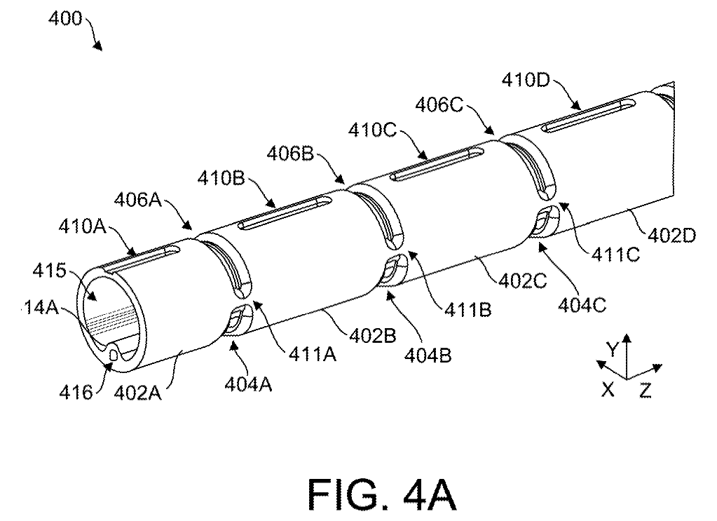

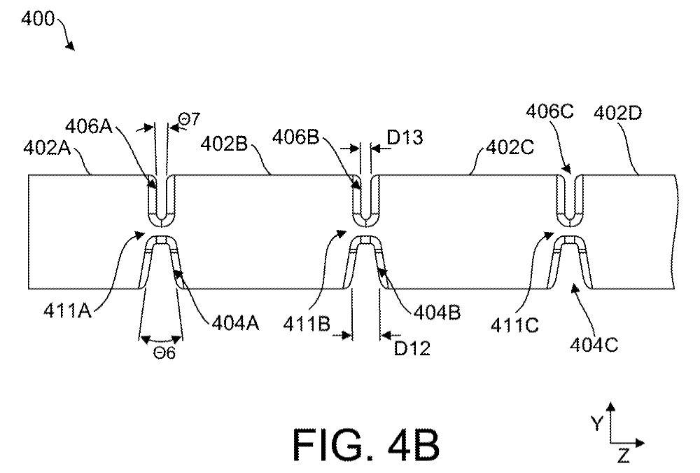

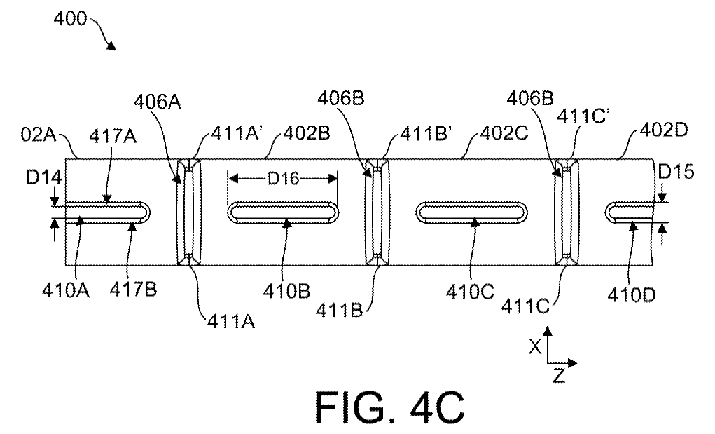

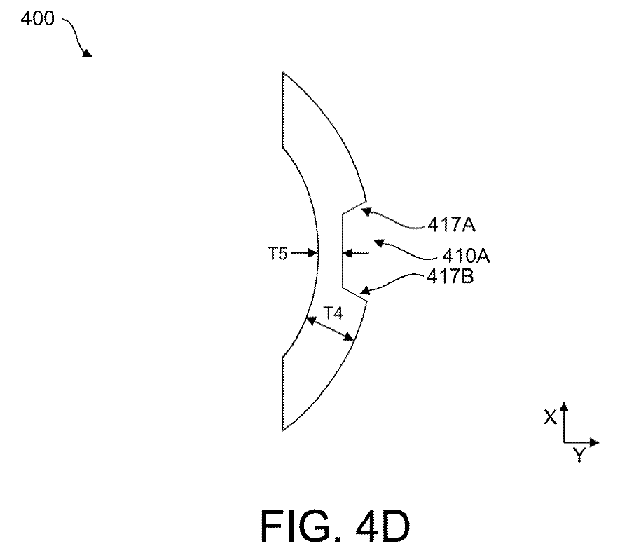

FIG. 4A is an enhanced, perspective view illustrating another exemplary articulation member 400, according to some embodiments of this disclosure. The articulation member 400 may be incorporated into the lead delivery catheter 100 (see FIG. 1). The articulation member 400 comprises a plurality of sections 402A, 402B, 402C, and section 402D. However, it will be appreciated that additional sections may be included in the articulation member 400. Each section of the sections 402A–402D defines a cavity 415 extending through the sections. The cavity 415 extends completely through the articulation member 400 along a direction parallel to the Z-axis to define or correspond to the catheter lumen 26 shown in FIG. 1.

The sections of the articulation member 400 are connected together via a plurality of joints, and these joints may be articulating joints in some embodiments. For example, section 402A is connected to section 402B via the joint 411A, section 402B is connected to section 402C via the joint 411B, section 402C is connected to section 402D via the joint 411C, and additional sections may similarly be connected via further joints. The articulation member 400 and other articulation members described herein are deflectable articulation members having articulating joints, and these deflectable articulation members may be smoothly used, positioned precisely, and used predictably. The joints and/or the articulation member as a whole may be molded components in some embodiments, but other alternative manufacturing approaches may be used — for example, tubing may be utilized, and slots may be cut in the tubing through laser cutting or mechanical cutting approaches. Other manufacturing techniques may also be used. While gaps are illustrated on both sides, gaps may be formed on only one side in some embodiments. For example, in some alternative embodiments, gaps on one side (e.g., gaps 404A–404C) may be provided and gaps on the opposing side (e.g., gaps 406A–406C) may be omitted.

As shown, each section of the articulation member 400 defines a thin wall portion that helps to facilitate cutting of the articulation member 400. For example, section 402A defines a thin wall portion 410A, section 402B defines a thin wall portion 410B, section 402C defines a thin wall portion 410C, and section 402D defines a thin wall portion 410D. While thin wall portion 410A extends to the extreme edge of section 402A in the negative Z-direction, sections 402B–402D each include end portions on opposite sides of the thin wall portions 410B–410D. The end portions may be beneficial to provide additional strength for the articulation member 400 and to help maintain the shape of the articulation member 400. Thin wall portions require the user to cut more to remove the articulation member, but thin wall portions are easier to cut relative to other articulation members without openings or thin wall portions. Thin wall portions also provided added strength relative to articulation members with openings, and thin wall portions also assist in maintaining the shape of the articulation members.

Further details regarding the articulation member 400 may be seen in FIGS. 4B. FIG. 4B is an enhanced, right-side view illustrating the articulation member of FIG. 4A, according to some embodiments of this disclosure. As can be seen in FIG. 4B, gaps are positioned on opposing sides of each of the joints. For example, gap 404A is positioned on a first side of the joint 411A, and gap 406A is positioned on a second side of the joint 411A. Gap 404B is positioned on a first side of the joint 411B, and gap 406B is positioned on a second side of the joint 411B. Gap 404C is positioned on a first side of the joint 411C, and gap 406C is positioned on a second side of the joint 411C. Gaps may be positioned similarly relative to other joints. The presence of these gaps allows articulation of one or more sections of the articulation member 400 about the joints. For example, section 402A may be rotated relative to section 402B, with section 402A rotating clockwise or counterclockwise relative to section 402B about a rotational axis that is parallel to the X-axis. Similar movement of different sections may also be accomplished.

The permitted amount of rotation may be more limited about rotational axes parallel to the Y-axis as the joints may be more resistant to rotation in this rotational direction. In order to accomplish rotation in a desired direction, the articulation member and/or the shaft within the articulation member may be rotated about the Z-axis relative to the patient, allowing the position of openings and joints to be adjusted. Thus, a wide variety of different shapes may be accomplished for the articulation member 400 and any shaft received therein.

The articulation member 400 is configured so that the sections may be articulated in two opposing directions, but the amount of possible articulation in one direction is less than the amount of possible articulation in the opposite direction. Each of the gaps 404A–404C generally define an angle ϴ6 when the articulation member 400 is in the resting position without any articulation of the articulation member 400 as shown in FIG. 4B. This angle ϴ6 is measured from the extreme wall of one section to the extreme wall of an opposing section. For example, at gap 404A, the angle ϴ6 is measured from the extreme wall of section 402A next to gap 404A to the extreme wall of section 402B next to gap 404A. Similarly, each of the gaps 406A–406C generally define an angle ϴ7 when the articulation member 400 is in the resting position without any articulation of the articulation member 400 as shown in FIG. 4B. This angle ϴ7 is measured from the extreme wall of one section to the extreme wall of an opposing section. For example, at gap 406A, the angle ϴ7 is measured from the extreme wall of section 402A next to gap 406A to the extreme wall of section 402B next to gap 406A.

In the articulation member 400, the angle ϴ6 and the angle ϴ7 are different. Larger values for angles ϴ6, ϴ7 may enable greater articulation as adjacent sections may rotate relative to each other in greater amounts without interfering with each other. Smaller values for angles ϴ6, ϴ7 may allow the amount of articulation in a particular direction to be more limited as adjacent sections may interfere with each other after a lesser amount of articulation, preventing further movement. Because angle ϴ6 is larger than angle ϴ7, the section 402A may be rotated relative to section 402B farther in the counterclockwise direction than the clockwise direction from the perspective shown in FIG. 4B.

Additionally, the gaps 404A–404C each define a distance D12. Distance D12 is measured from points at the extreme edges of adjacent sections, with these points being positioned at the farthest points of the sections along the negative Y-direction. Similarly, gaps 406A–406C each define a distance D13. Distance D13 is measured from points at the extreme edges of adjacent sections, with these points being positioned at the farthest points of the sections along the positive Y-direction. In the articulation member 400, the distances D12 and D13 are different, with distance D12 being larger than distance D13. However, the distances may be different in other embodiments. By changing the distances D12, D13, the amount of possible articulation may be adjusted. For example, lower distances may tend to limit the amount of possible articulation, and larger distances may tend to increase the amount of possible articulation.

The surfaces of the sections proximate to thin wall portions may also be tapered, and this taper may help ensure that cutting occurs along the thin wall portions. For example, in FIG. 4E, a first surface 417A and the second surface 417B are positioned on opposing sides of the thin wall portion 410A, with the first surface 417A positioned in the positive X-direction relative to the second surface 417B. Surfaces 417A, 417B each define a taper angle similar to taper angle ϴ1’ in the articulation member 100 of FIG. 1E. This taper angle for surfaces 417A, 417B may be about 30 degrees in the articulation member 400, but different taper angles may be used in other embodiments. Alternatively, articulation members may not have tapered surfaces adjacent to openings in other embodiments. The surface 417A and the surface 417B may be separated by a minimum distance D14. The minimum distance D14 determines the thickness of the thin wall section in the X-direction 408A and determines how much the direction of cutting is controlled. The distance D15 is the maximum distance between the surface 417A and surface 417B, and larger values for distance D15 may be beneficial so that higher taper angles may be accomplished. The minimum distances D14 and D15 may be have a range of values depending on the particular structural and functional needs of the articulation member 400.

The articulation member 400 also defines extended portions at each of the sections, with an extended portion 414A positioned at the section 402A and others positioned at other sections. The extended portions project into the cavity 415. The extended portions may contact a shaft extending within the cavity 415 in some embodiments to constrain the motion of the shaft.

A lumen 416 is also defined within the extended portion of each of the sections of the articulation member 400. The lumen 416 may be configured to receive a steering element, and tension may be applied to this steering element to cause the shape of the articulation member 400 to be adjusted. In some embodiments, the application of tension on a wire may cause similar amounts of articulation at each of the joints of the articulation member, but the amount of articulation in the various joints may differ in other embodiments. In some embodiments, extended portions defining lumens 416 are only positioned at the sections at opposing ends of the articulation member, and a wire may extend through the lumen at one end section, through the cavity to the opposing end section, and out of the lumen at the opposing end section. However, in other embodiments, extended portions defining lumens 416 may be positioned at one or more sections. In the illustrated embodiment of FIGS. 4A, the extended portions defining lumens 416 are positioned at each of the sections within the articulation member 400.

FIG. 4C is a top view illustrating the articulation member of FIG. 4A, according to some embodiments of this disclosure. In this top view, the joint 411A’, the joint 411B’, and the joint 411C’ are visible. Each of joints 411A’–411C’ are positioned at the extreme position of the articulation member 400 along the positive X-direction. Joint 411A’ connects section 402A to section 402B, joint 411B’ connects section 402B to section 402C, and joint 411C’ connects section 402C to section 402D. Joints 411A’–411C’ may operate similarly to joints 411A–411C.

Additionally, FIG. 4C allows further details regarding the thin wall portions to be seen. Other than thin wall portions positioned on sections located at extreme ends of the articulation member 400 (e.g., thin wall portions 410A at section 402A), the thin wall portions generally define a length D16 along a direction parallel to the Z-axis. The length D16 may vary in different embodiments. Furthermore, the thin wall portions may be sized in different proportions relative to the sections. For example, the length of thin wall portion 410B may be made smaller to reduce the size of thin wall portion 410B relative to the section 402B.

FIG. 4D is a cross-sectional view illustrating a portion of a cross section at a thin wall portion in the articulation member of FIG. 4A, according to some embodiments of this disclosure. The articulation member 400 defines a thickness T4 at portions of the articulation member 400 away from the thin wall portions and the extended portions. The articulation member 400 also defines a minimum thickness T5 at the thin wall portions. The minimum thickness T5 may be less than the thickness T4, with thickness T5 being about 90% of the thickness T4 or lower, about 75% of the thickness T4 or lower, about 50% of the thickness T4 or lower, or even about 25% of the thickness T4 or lower. The reduced size of minimum thickness T5 may enable the articulation member 400 to be cut more easily, but a larger minimum thickness T5 may be beneficial to provide increased strength for the articulation member 400.

In order to fully cut the articulation member, a user is required to cut more portions of the articulation member 400 as compared to articulation members 100, 200, and 300. However, thin wall portions may still allow cutting to be performed more easily than other articulation members without openings or thin wall portions. Articulation members 100, 200, and 300 have openings at each of the sections to reduce the amount of cutting required. By contrast, a user must cut through the thin wall portions in articulation member 400. However, the thin wall portions provide added strength for the articulation member 400 relative to articulation members 100, 200, and 300, and the articulation member 400 may better maintain its shape relative to articulation members 100, 200, and 300 due to the presence of the thin wall portions.

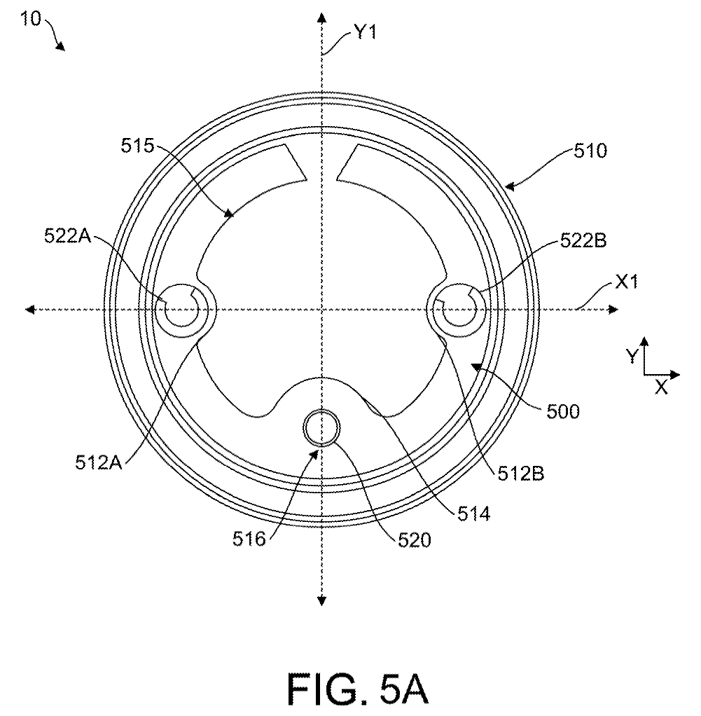

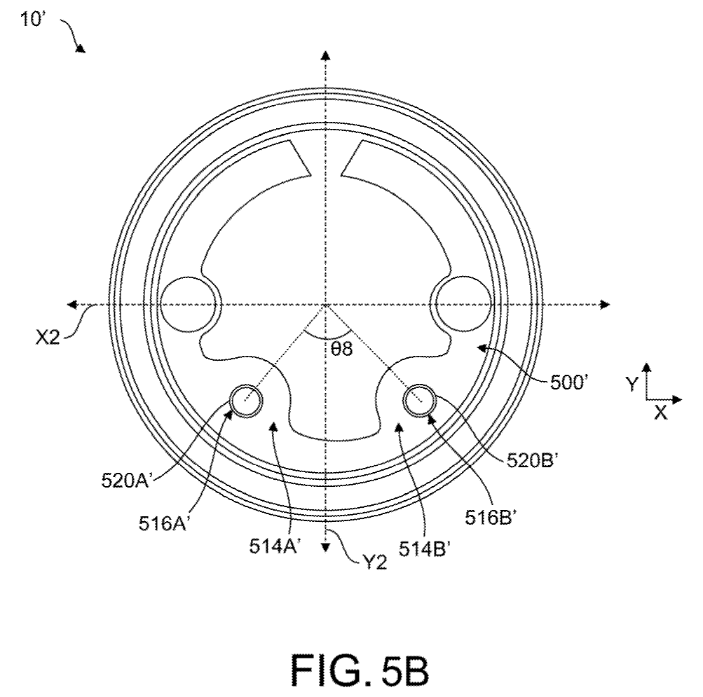

FIG. 5A is cross-sectional view of an embodiment of the deflection region 20 of the shaft 14 of the lead delivery catheter 10 of FIG. 1, including an articulation member 500 disposed therein. As shown, the shaft 14 in the deflection region 20 includes an outer jacket 510 disposed over the articulation member 500. In embodiments, the outer jacket 510 may be a braided tube as is commonly used in medical device catheter construction. As the skilled artisan will appreciate, a braided outer jacket or covering may readily be cut using a conventional catheter slitting device during removal of the catheter 10 after lead implantation. Alternatively, the outer jacket 510 may be a non-reinforced polymeric layer, e.g., an unbraided tube or an overmolded material. In the illustrated embodiment, the articulation member 500 has the same general configuration as the articulation member 200 described previously, although in other embodiments the articulation members 300 or 400 could be readily incorporated.

As shown, the articulation member 500 defines a primary lumen 515 and also includes an auxiliary lumen 516 as in the other embodiments described previously, and FIG. 5A illustrates a steering element 520 disposed in the auxiliary lumen 516. As discussed previously, the steering element 520 is operatively connected to an actuator in the catheter handle (see FIG. 1) and is also anchored to the shaft 14 (FIG. 1) distal to the articulation member 500. Accordingly, operation of the actuator applies a deflection force to the steering element 520 to cause the shaft to deflect in the plane Y1 shown in FIG. 5A.

As further shown, the articulation member 500 further includes reinforcing elements 522A and 522B disposed, respectively, within protrusions 512A and 512B, which correspond to the joints between adjacent sections of the articulation member 500 as described above. When present, the reinforcing elements 522A, 522B operate to maintain planarity of deflection, i.e., by resisting deflection in the plane X1 when the shaft is deflected in the plane Y1 under the action of the steering element 520. however, in other embodiments, the reinforcing elements 522A, 522B are omitted.

An alternative embodiment of the delivery catheter 10’ shown in cross-section in FIG. 5B, includes an alternative articulation member 500’ that is in most respects similar to the articulation member 500, except that the articulation member 500’ includes two auxiliary lumens 516A’, 516B’ disposed within respective extended portions 514A’, 514B’. Additionally, steering elements 520A’ and 520B’ are disposed, respectively, within auxiliary lumens 516A’ and 516B’.

Extended portion 514A’ is spaced apart from the extended portion 514B’. The articulation member 500’ defines an axis X2 that is parallel to the X-axis, and the articulation member 500’ also defines an axis Y2 that is parallel to the Y-axis. The axis X2 and the axis Y2 intersect at a center point that is located at a center of the shape formed by the outer diameter of the articulation member 500’, and angles may be measured relative to this center point in the X-Y plane. The center of the lumen 516A’ defined in the extended portion 514A’ is positioned away from the center of the lumen 516B’ defined in the extended portion 514B’ by an angle ϴ8, and this angle ϴ8 is about 90 degrees. However, the extended portions 514A’, 514B’ and lumens 516A’, 516B’ may be positioned at different locations on a articulation member in other embodiments. A different number of lumens may be used (e.g., 3, 4, or even more).

The use of two steering elements may be beneficial to accomplish more complex curvatures for the articulation member 500’ and any shaft received therein. Tension may be applied to both of the wires simultaneously, and the amount of tension applied to each of the wires may or may not be different. The ability to apply tension at multiple wires allows the forces to be more evenly dispersed, and less tension may be necessary at each wire. The positioning of the lumens 516A’, 516B’ may beneficially allow rotation about an axis parallel to the X-axis, and the positioning of the lumens 516A’, 516B’ may beneficially allow some rotation about an axis parallel to the Y-axis. Lumens may be positioned at alternative locations on the articulation member such as at portions of the articulation member 500 between the lumen 523A and the top opening or at portions of the articulation member 500’ between the lumen 523B and the top opening.

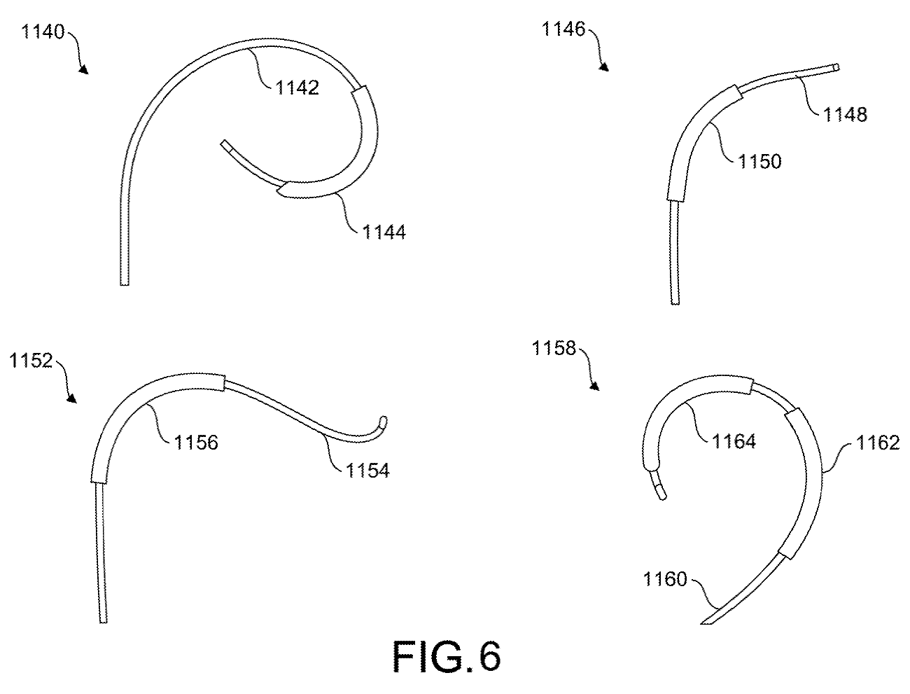

FIG. 6 is a schematic view illustrating exemplary deflection region shapes for the lead delivery catheters utilizing the various articulation members 100, 200, 300, 400 and 500 as previously described. For illustration purposes, the respective deflection regions are shown as enlarged in FIG. 6, but the skilled artisan will readily recognize that in the actual delivery catheter the shaft in the deflection region is substantially isodiametric with the remainder of the shaft. In delivery catheter 1140, a shaft 1142 is illustrated with one deflection region 1144 positioned along the shaft 1142. In delivery catheter 1146, a shaft 1148 is illustrated with one deflection region 1150 positioned on the shaft 1148. In delivery catheter 1152, a shaft 1154 is illustrated with one deflection region 1156 positioned along the shaft 1154. In delivery catheter 1158, a shaft 1160 is illustrated with a first deflection region 1162 and a second deflection region 1164 positioned at different locations along the shaft 1160. As illustrated in FIG. 6, the shafts and deflection regions may each be adjusted into a wide variety of shapes based on the location, configuration, and number of articulation members utilized. In some embodiments, the assemblies, shafts, and articulation members may be pre-curved so that the articulation member generally does not extend along a straight line when no tension is applied to the lead delivery articulation member, and this may be beneficial where an assembly is being used for a specific purpose where curvature is necessary. However, in other embodiments, the assemblies, shafts, and articulation members are not pre-curved, and the lead delivery articulation member generally extends along a straight line when no tension is applied to the lead delivery articulation member. The assemblies, shafts, and articulation members may be adjusted in shape by applying tension via a wire or using other techniques.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A cuttable articulating catheter assembly comprising:
a handle member;
a catheter shaft extending from the handle member and including an articulation member configured selectively articulate in response to a deflection force, the articulation member including:
a plurality of tubular sections, and a plurality of joints, wherein each joint of the plurality of joints is positioned between two adjacent tubular sections of the plurality of tubular sections, and wherein a first gap and a second gap are positioned between adjacent sections of the plurality of sections, the first gap positioned on a first side of each joint of the plurality of joints, and the second gap positioned on a second side of each joint of the plurality of joints, and
wherein each tubular section includes a cutability feature configured to facilitate cutting of the catheter shaft member along the articulation member.

2. The cuttable articulating catheter assembly of claim 1, wherein the cutability feature defines a length extending in a first direction that is parallel to a lengthwise direction of the articulation member, and the cutability feature is configured to guide a cutter so that the cutter cuts the articulation member along the lengthwise direction of the articulation member.

3. The cuttable articulating catheter assembly of claim 2, wherein the cutability feature is an opening in the form of a slot.

4. The cuttable articulating catheter assembly of claim 2, wherein the cutability feature is a thin wall portion that has a reduced wall thickness relative to other portions of a wall on a respective section.

5. The cuttable articulating catheter assembly of claim 2, wherein the articulation member includes a first auxiliary lumen extending through at least two tubular sections of the plurality of tubular sections.

6. The cuttable articulating catheter assembly of claim 1, further comprising a first steering element disposed in the first auxiliary lumen, wherein the first steering element is configured to receive a first tension force to selectively deflect the articulation member.

7. The cuttable articulating catheter assembly of claim 6, wherein the articulation member generally extends along a straight line when no tension is applied to the lead delivery articulation member via the first steering element.

8. The cuttable articulating catheter assembly of claim 6, wherein the articulation member includes a second auxiliary lumen extending through the at least two tubular sections.

9. The cuttable articulating catheter assembly of claim 8, further comprising a second steering element disposed in the second auxiliary lumen, wherein the second steering element is configured to receive a second tension force to selectively deflect the articulation member.

10. The cuttable articulating catheter assembly of claim 9, wherein articulation member defines a central axis extending in a lengthwise direction, wherein the lengthwise direction is orthogonal to a first plane, wherein the first auxiliary lumen and the second auxiliary lumen are positioned less than about 180 degrees apart from each other relative to the central axis in the first plane.

11. The cuttable articulating catheter assembly of claim 10, wherein the first auxiliary lumen and the second auxiliary lumen are positioned less than about 90 degrees apart from each other relative to the central axis in the first plane.

12. An articulation member for a deflectable lead delivery catheter, the articulation member comprising:
a plurality of longitudinally spaced tubular sections; and
a plurality of joints, wherein each joint of the plurality of joints is positioned between two adjacent tubular sections of the plurality of tubular sections,
wherein a first gap and a second gap separate adjacent tubular sections on opposite sides of the joints positioned therebetween, and
wherein each tubular section includes a cutability feature configured to facilitate cutting of the articulation member along its length.

13. The articulation member of claim 12, wherein the cutability feature defines a length extending in a first direction that is parallel to a lengthwise direction of the articulation member, and the cutability feature is configured to guide a cutter so that the cutter cuts the articulation member along the lengthwise direction of the articulation member.

14. The articulation member of claim 13, wherein the cutability feature is an opening in the form of a slot.

15. The articulation member of claim 13, wherein the cutability feature is a thin wall portion that has a reduced wall thickness relative to other portions of a wall on a respective section.

16. The articulation member of claim 13, wherein the articulation member includes a first auxiliary lumen extending through at least two tubular sections of the plurality of tubular sections, the first auxiliary lumen being dimensioned to slidably receive a steering element.

17. A cuttable articulating catheter assembly comprising:
a handle member;
a catheter shaft extending from the handle member and including an articulation member configured selectively articulate in response to a deflection force, the articulation member including:
a plurality of longitudinally spaced tubular sections; and
a plurality of joints, wherein each joint of the plurality of joints is positioned between two adjacent tubular sections of the plurality of tubular sections,
wherein a first gap and a second gap separate adjacent tubular sections on opposite sides of the joints positioned therebetween, and
wherein each tubular section includes a cutability feature, wherein the cutability feature defines a length extending in a first direction that is parallel to a lengthwise direction of the articulation member, and the cutability feature is configured to guide a cutter so that the cutter can cut the shaft along the lengthwise direction of the articulation member.

18. The lead delivery catheter of claim 17, wherein the cutability feature is an opening in the form of a slot.

19. The lead delivery catheter of claim 17, wherein the cutability feature is a thin wall portion that has a reduced wall thickness relative to other portions of a wall on a respective section.

20. The lead delivery catheter of claim 17, wherein the articulation member includes a first auxiliary lumen extending through at least two tubular sections of the plurality of tubular sections, and wherein the lead delivery catheter further comprises comprising a first steering element disposed in the first auxiliary lumen, wherein the first steering element is configured to receive a first tension force to selectively deflect the articulation member first auxiliary lumen being dimensioned to slidably receive a steering element.
